(19) United States
(12) Patent Application Publication
(10) Pub. No.: US 2014/0275721 A1
(43) Pub. Date: Sep. 18, 2014

(54) Centrifugal Blood Pump With Partitioned Implantable Device

(71) Applicant: THORATEC CORPORATION, Pleasanton, CA (US)

(72) Inventors: Masamichi Yanai, Ann Arbor, MI (US); Brian D. Jeffery, Melvindale, MI (US)

(73) Assignee: THORATEC CORPORATION, Pleasanton, CA (US)

(21) Appl. No.: 13/804,144

(22) Filed: Mar. 14, 2013

(51) Int. Cl. A61M 1/10

(52) U.S. Cl. CPC A61M 1/1012
USPC 600/16

(57) ABSTRACT

A centrifugal blood pump system has a self-contained pumping unit and a self-contained motor unit. A pump outlet extends radially from a pump housing of the pumping unit. A percutaneous cable passes through a radial exit from a motor housing of the motor unit. The motor housing has a substantially planar face configured to mate with a substantially planar face of the pump housing. The pumping unit and the motor unit are configured to latch together in a plurality of orientations, each orientation having the substantially planar faces mated and the outlet and radial exit at a different respective angular separation. The pumping unit and the motor unit are configured such that after the pumping unit is implanted, the motor unit can be unlatched and a replacement motor unit latched with the pumping unit at the plurality of orientations.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to circulatory assist devices, and, more specifically, to an implanted device for a pumping system partitioned into separable pumping and motor units.

Many types of circulatory assist devices are available for either short term or long term support for patients having cardiovascular disease. For example, a heart pump system known as a left ventricular assist device (LVAD) can provide long term patient support with an implantable pump associated with an externally-worn pump control unit and batteries. The LVAD improves circulation throughout the body by assisting the left side of the heart in pumping blood. One such system is the DuraHeart® LVAS system made by Terumo Heart, Inc., of Ann Arbor, Mich. The DuraHeart® system employs a centrifugal pump with a magnetically levitated impeller to pump blood from the left ventricle to the aorta. An electric motor magnetically coupled to the impeller is driven at a speed appropriate to obtain the desired blood flow through the pump.

A typical cardiac assist system includes a pumping unit, electrical motor (e.g., a brushless DC motor integrated in the pump housing), drive electronics, microprocessor control unit, and an energy source such as rechargeable batteries and/or an AC power conditioning circuit. The system is implanted during a surgical procedure in which a centrifugal pump is placed in the patient's chest. An inflow conduit is pierced into the left ventricle to supply blood to the pump. One end of an outflow conduit is mechanically fitted to the pump outlet and the other end is surgically attached to the patient's aorta by anastomosis. A percutaneous cable connects to the pump, exits the patient through an incision, and connects to the external control unit.

The goal of the control unit is to autonomously control the pump performance to satisfy the physiologic needs of the patient while maintaining safe and reliable system operation. A control system for varying pump speed to achieve a target blood flow based on physiologic conditions is shown in U.S. Pat. No. 7,160,243, issued Jan. 9, 2007, which is incorporated herein by reference in its entirety. A target blood flow rate may be established based on the patient's heart rate so that the physiologic demand is met. The control unit may establish a speed setpoint for the pump motor to achieve the target blood flow.

A typical pump motor employed for a blood pump is a three-phase permanent magnet electric motor that can be driven as a brushless DC or a synchronous AC motor without any position sensor. The need for a position sensor is avoided by controlling motor operation with one of a variety of methods that use the measured stator phase currents to infer the position. Vector control is one typical method used in variable frequency drives to control the torque and speed of a three-phase electric motor by controlling the current fed to the motor phases. This control can be implemented using a fixed or variable voltage drive delivered via an inverter comprised of pulse width modulated H-bridge power switches arranged in phase legs.

Reliability, fault detection, and fault tolerance are important characteristics of an electrically-powered blood pump, drive system, and cable. Co-pending application U.S. Ser. No. 13/418,447, filed Mar. 13, 2012, entitled “Fault Monitor For Fault Tolerant Implantable Pump,” which is hereby incorporated by reference, discloses a fault-tolerant inverter/cable system wherein redundant inverter legs are coupled to the motor phases by redundant, parallel conductors between the external unit and the implanted pump. For a three-phase motor, the redundant interconnect system includes six conductors in the cable. By monitoring the equality of the current and/or voltage of the two conductors on the same phase, a fault or impending fault can be detected for each individual conductor. Co-pending application U.S. Ser. No. 13/742,469, filed Jan. 16, 2013, entitled “Motor Fault Monitor for Implantable Blood Pump,” which is hereby incorporated by reference, discloses technology for detecting other pump failures such as a soldering terminal failure, a coil wire breakage, damage to a flex circuit substrate, a coil turn-to-turn short, a layer-to-layer short, and a core/yoke detachment.

The conventional pumping unit for an implanted system has employed a hermetically sealed housing containing the elements of the pump and motor (i.e., the housing body includes a pumping chamber for containing the impeller and one or more other chambers for containing the motor, magnetic components, and electronics). In the event of a fault associated with any one of the pumping chamber, impeller, motor, magnetic components, or electronics that is serious enough to require replacement, then a surgical explantation procedure is performed in which the pumping unit is detached from the inflow and outflow conduits and then removed. A replacement unit is then implanted and attached to the existing conduits or conduits the conduits may sometimes also be replaced. It would be desirable to reduce the invasiveness of such surgical replacement procedures.

With an integrated housing containing an inlet and an outlet for the pumping chamber and a connector/cable exit, the angular separation between the direction in which the outlet extends and the direction in which the cable exits is fixed by the housing design. A nominal angle has been chosen that provides an optimal placement for a person having an average physiology. However, structural differences in the physiology of individual patients may present obstructions that could be avoided if the cable exited at an angular separation from the outlet other than at the conventional fixed position.

### SUMMARY OF THE INVENTION

In one aspect of the invention, a centrifugal blood pump system is provided for implanting into a patient. A self-contained pumping unit comprises a pump housing having an inlet, an outlet, and a pump chamber, and an impeller disposed in the pump chamber. The inlet extends axially from the pump housing on an inlet side of the pump housing, and the outlet extends radially from the pump housing. The pump housing has a substantially planar face opposite from the inlet side. A self-contained motor unit comprises a motor housing, a motor stator disposed in the motor housing, and a percutaneous cable passing through a radial exit from the motor housing. The motor housing has a substantially planar face configured to mate with the substantially planar face of the pump housing. The pumping unit and the motor unit are configured to latch together in a plurality of orientations, each orientation having the substantially planar faces mated and the outlet and radial exit at a different respective angular separation. The pumping unit and the motor unit are configured such that after the pumping unit is implanted, the motor unit can be unlatched and a replacement motor unit latched with the pumping unit at the plurality of orientations.

### BRIEF DESCRIPTION OF THE DRAWINGS

FIG. 1 is a diagram of a circulatory assist system as one example of an implantable pump employing the present invention.

FIG. 2 is a perspective view of a prior art centrifugal pump with a fixed angular separation between the outlet and the cable.

FIG. 3 is a cross section showing one embodiment of a partitioned implantable device with separate pumping and motor units.

FIG. 4 is a perspective view showing a plurality of orientations between the outlet and the cable exit.

FIG. 5 is an exploded view showing another embodiment of partitioned pumping and motor units.

FIG. 6 shows another embodiment of the invention using a clip to attach the partitioned units.

FIG. 7 shows another embodiment of the invention using a radial collar and pin to attach the partitioned units.

FIG. 8 shows another embodiment of the invention using a radial collar and buckle to attach the partitioned units.

### DETAILED DESCRIPTION OF PREFERRED EMBODIMENTS

Referring to FIG. 1, a patient 10 is shown in fragmentary front elevational view. Surgically implanted either into the patient's abdominal cavity or pericardium 11 is the pumping/motor unit 12 of a ventricular assist device. An inflow conduit (on the hidden side of unit 12) pierces the heart to convey blood from the patient's left ventricle into pumping unit 12. An outflow conduit 13 conveys blood from pumping unit 12 to the patient's aorta. A percutaneous power cable 14 extends from pumping unit 12 outwardly of the patient's body via an incision to a compact control unit 15 worn by patient 10. Control unit 15 is powered by a main battery pack 16 and/or an external AC power supply and an internal backup battery. Control unit 15 includes a commutator circuit for driving a motor within pumping unit 12. Control unit 15 monitors for various faults that may occur in pumping/motor unit 12 and generates an alarm whenever a fault is detected that requires correction, by surgical replacement or otherwise.

FIG. 2 is a perspective view of a prior art pumping/motor unit 20 connected to an inflow conduit 21. Unit 20 has an outlet 22 adapted to be coupled to an outflow conduit or graft 23. An electrical cable 24 exits unit 20 via a cable exit 25 (which may include an electrical connector, not shown). In a typical integrated pump and motor housing, the fixed angular separation between outlet 22 and cable exit 25 has been relatively small (e.g., between 0° and 90°). In one aspect of the invention, surgical placement of both the outflow conduit and the electrical cable can be improved by providing an adjustable angular separation that allows an implantation to best conform to the physiology of the patient.

FIG. 3 shows a centrifugal pump device 30 having a self-contained pumping/impeller unit 31 and a self-contained motor unit 32. Each self-contained unit is hermetically sealed to preventingress of tissue or fluids other than via an inlet 33 and an outlet 34 in a pump housing 35 of unit 31. Pump housing 35 has a hollow, generally cylindrical or puck shape. Inlet 33 extends axially from an inlet side of pump housing 35. Outlet 34 extends from a radial edge of pump housing 35 at a predetermined exit point defined by a volute of a pumping chamber 37. Outlet 34 is typically oriented tangentially at the radial exit point. An impeller 36 resides within pumping chamber 37 over a hub 38. Impeller 36 may include upper and lower plates containing embedded magnets (40 in the upper plate and 41 in the lower plate) and sandwiched over a plurality of vanes 42. Embedded magnets 40 interact with a levitating magnetic field created by levitation magnets 43 disposed in pump housing 35. Embedded magnet segments 41 in the lower plate of impeller 36 magnetically couple with a rotating magnet field generated in motor unit 32 in order to spin impeller 36 and thereby pump blood out through outlet 34.

Motor unit 32 has a motor housing 50 containing a multi-phase motor stator 45 which includes windings 46 and 47 and respective magnetic cores 48 and 49. Although a stationary stator which couples with the impeller is shown (i.e., wherein impeller 36 is directly driven as a rotor of the stationary stator), motor unit 32 can alternatively carry a spinning rotor which carries permanent magnets on the rotor for magnetically driving impeller 36 as known in the art. Housings 35 and 50 may be comprised of biocompatible thermoplastics.

A cable portion 51 on motor unit 32 enters/exits at a radial cable exit feature 52. Electrical conductor 53 connects to windings 46 and 47 to supply electrical power for operating stator 45. An electrical connector (not shown) may be located within radial exit 52. With or without a connector, radial exit 52 is sealed against ingress of fluids or tissues.

Pump housing 35 has a substantially planar face 55 on the side opposite from the inlet side. Motor housing 50 has a substantially planar face 56 configured to mate with planar face 55. A raised collar 57 extends around the periphery of planar face 56 so that pumping unit 31 and motor unit 32 can be brought together in a nested relationship. Preferably, both planar faces 55 and 56 are circular so that the units can be nested together in any rotated orientation, i.e., with pump outlet 34 and radial cable exit 52 at any radial positions with any desired angular separation.

To ensure proper control of the rotation of impeller 36, planar faces 55 and 56 must be intimately attached according to the desired relative positioning. In the embodiment shown in FIG. 3, units 31 and 32 are latched together magnetically via an embedded magnet 60 just behind planar face 55 in pump housing 35 and an embedded magnet 61 just behind planar face 56 in motor housing 50. With units 31 and 32 being axially pressed together, collar 57 prevents any undesirable radial movement of the units. The magnetic attraction between magnets 60 and 61 is sufficient to ensure mating of faces 55 and 56 during implantation and use within the patient, but can be manually overcome when desired so that a defective or faulted motor unit can be easily removed during a surgery being conducted to replace it. Due to the partitioning of the implanted device between self-contained pumping and motor units, the pump inflow and outflow connections are undisturbed in the event that the failure or defect resides only in the motor unit and not the pumping unit. Thus, the surgical procedure is much less invasive. When the replacement unit is latched with the existing pumping unit, the cable exit can be positioned in any radial orientation so that the best available placement can be utilized.

FIG. 4 is a perspective, exploded view showing a self-contained pumping unit 63 attached to the apex of a heart 64 (via an inflow conduit and apical cuff, not shown). An outlet 65 can be oriented at any desired radial position during implantation (with a different radial position shown by dashed lines). A self-contained motor unit 66 can be mated to pumping unit 63 with a radial cable exit 67 located at any arbitrary radial position. Alternative radial positions of cable exit 67 being shown by dashed lines. Units 63 and 66 incorporate a latching mechanism 68/69, e.g., a magnetic latch, that is effective at any rotated position. As an alternative to a magnetic latch, a screw connection or other similar fastener can be used as shown in the next embodiment.

FIG. 5 shows a pumping unit 70 with an axial inlet 71 and a radial outlet 72. A planar face 73 of unit 70 has a regular, noncircular shape with a plurality of rotational symmetries. In the illustrated embodiment, the shape is octagonal which results in eight different symmetrical positions. A motor unit 74 has a radial cable exit 75 and flexible cable 76 for connecting to an external control unit (not shown). A planar face 77 is surrounded by a peripheral collar 78 defining a closed, noncircular path having the same rotational symmetries as face 73. Thus, face 73 of pumping unit 70 is keyed with collar 78 so that the faces can be mated in one of the plurality of symmetrical orientations. Collar 78 prevents both radial (i.e, planar) side-to-side movement and rotational movement between the faces. Pumping unit 70 has a threaded fastening hole 80 and motor unit 74 has a fastening hole 81 that are aligned in parallel when faces 73 and 77 are mated. A screw 82 has an elongated member 83 with a threaded shaft 84 at one end for entering hole 80 and a flanged portion 85 at the other end for bearing against motor unit 74.

FIG. 6 shows an alternative embodiment using a clip. A self-contained pumping unit 86 has an inlet 87 and an radial outlet 88. A planar face 89 surrounded by a collar 90 is directed toward a self-contained motor unit 91. A radial cable exit 92 extends from motor unit 91. A planar face 93 is directed toward face 89 and may be surrounded by a ledge 94 that receives collar 90. Collar 90 and ledge 94 define a periphery that is circular or has any other shape that includes a plurality of rotational symmetries. A circular shape is most preferred since it conforms to the shape of the impeller and impeller chamber, and, consequently, provides the smallest size. After faces 89 and 93 are brought together with any desired angular separation between outlet 88 and cable exit 92, they are held together by a C-shaped clip 95 having a main webbing 96 extending between a bottom member 97 and a top member 98. Bottom member 97 has an outer ridge 100 for capturing motor unit 91. Top member 98 defines a U-shaped slot 101 between fingers 102 and 103. Fingers 102 and 103 extend over pumping unit 86 so that outlet 87 is disposed in slot 101. Clip 95 is comprised of a material that is sufficiently flexible to allow members 97 and 98 to spring apart in order to install on or be removed from units 86 and 91.

FIG. 7 shows an alternative embodiment wherein a pumping unit 105 has an axial inlet 106 and a radial outlet 108. A semi-circular collar 108 has a groove 110 defined by a lip 11 that extends radially inward. Collar 108 extends for no more than 180° so that it can receive a flange 117 that extending from one side of a motor unit 115. Motor unit 115 has a cable exit 116 and a circumferential groove 188 that defines flange 117. Groove 118 and flange 117 extend completely around motor unit 115 so that flange 117 can be inserted into groove 110 with cable exit 116 in any desired orientation.

Flange 117 slides radially into groove 110. A capture mechanism is comprised of a sliding pin 121 received in a pocket 120 in pumping unit 105 that is placed radially outward from the mated planar faces on the opposite side from the semicircular collar. The capture mechanism has a capture member with an cam end 122 with an angled cam surface at one end and a knob 123 at the other end. A spring 124 is disposed between pocket 120 and cam end 122 so that end 122 is urged downward. When flange 117 is being slid into groove 110, motor unit 115 forces cam end 122 into the pocket against spring 124. Once flange 117 fully enters groove 110 then cam end 122 extends downward so that motor unit 115 is captured on pumping unit 105 with their planar faces securely fastened until a manual pull on knob 123 releases them.

FIG. 8 shows a modified embodiment wherein the capture member is comprised of a buckle 130 instead of the sliding pin of FIG. 7. Thus, motor unit 115 has a plurality of hooks 133 spaced around its periphery. After inserting flange 117 into groove 110, one of hooks 133 is lined up with a lever 131 and ring 132 of buckle 130. The selection of a hook 133 places a cable exit (not shown) at one of a predetermined number of orientations with a respective angular separation between the pump outlet and the cable exit.

What is claimed is:

1. A centrifugal blood pump system for implanting into a patient, comprising:
a self-contained pumping unit comprising a pump housing having an inlet, an outlet, and a pump chamber, and an impeller disposed in the pump chamber, wherein the inlet extends axially from the pump housing on an inlet side of the pump housing, wherein the outlet extends radially from the pump housing, and wherein the pump housing has a substantially planar face opposite from the inlet side; and
a self-contained motor unit comprising a motor housing, a motor stator disposed in the motor housing, and a percutaneous cable passing through a radial exit from the motor housing, wherein the motor housing has a substantially planar face configured to mate with the substantially planar face of the pump housing;
wherein the pumping unit and the motor unit are configured to latch together in a plurality of orientations, each orientation having the substantially planar faces mated and the outlet and radial exit at a different respective angular separation; and
wherein the pumping unit and the motor unit are configured such that after the pumping unit is implanted the motor unit can be unlatched and a replacement motor unit latched with the pumping unit at the plurality of orientations.

2. The system of claim 1 wherein at least one of the pumping unit and motor unit includes a collar along a periphery of the respective substantially planar face for establishing a predetermined alignment of the impeller and the motor stator.

3. The system of claim 2 wherein the collar is comprised of a semicircular extension from a first one of the pumping unit or the motor unit, wherein the semicircular extension includes a circumferential groove on an interior side, and wherein the other one of the pumping unit or motor unit includes a flange configured to be received in the groove.

4. The system of claim 3 wherein the flange slides radially into the groove, and wherein the system further comprises a movable capture member disposed radially outward from the mated substantially planar faces to prevent radial movement between the pumping unit and the motor unit after the flange enters the groove.

5. The system of claim 4 wherein the capture member is comprised of a sliding pin.

6. The system of claim 4 wherein the capture member is comprised of a buckle.

7. The system of claim 1 wherein the pumping unit and motor unit each includes a latching magnet, wherein the latching magnets are configured to provide an attractive force for urging together the substantially planar faces.

8. The system of claim 1 wherein the pumping unit and motor unit each includes a fastening hole, wherein the fastening holes are parallel when the impeller and the motor stator have a predetermined alignment, and wherein the system further comprises a fastener disposed in the fastening holes to attach the pumping unit and the motor unit.

9. The system of claim 1 further comprising a C-shaped clip disposed over the pumping unit and the motor unit.

10. The system of claim 1 wherein at least one of the pumping unit and motor unit includes a collar along a periphery of the respective substantially planar face for establishing a predetermined alignment of the impeller and the motor stator, wherein the collar defines a closed, noncircular path having a plurality of rotational symmetries, and wherein the other one of the pumping unit and motor unit has a perimeter shape around the respective substantially planar face that is keyed to the path of the collar.
